(19) United States
(12) Patent Application Publication
(10) Pub. No.: US 2023/0218744 A1
(43) Pub. Date: Jul. 13, 2023

(54) HYPERIMMUNE IGG AND/OR IGM COMPOSITIONS AND METHOD FOR PREPARING THEREOF AND METHOD FOR OBTAINING HYPERIMMUNE HUMAN PLASMA FROM A DONOR

(71) Applicant: GRIFOLS WORLDWIDE OPERATIONS LIMITED, DUBLIN (IE)

(72) Inventors: Peter VANDEBERG, Dublin (IE); Andrea WELLHOUSE, Dublin (IE); Marta JOSE, Dublin (IE); Marilyn ROSA-BRAY, Dublin (IE); Charles AUGER, Dublin (IE); Rodrigo GAJARDO RODRÍGUEZ, Dublin (IE); Myles LINDSAY, Dublin (IE); Natalia ORLOVA, Dublin (IE); Erin ROMES, Dublin (IE)

(21) Appl. No.: 18/000,380

(22) PCT Filed: May 26, 2021
(86) PCT No.: PCT/EP2021/063995

§ 371 Date: Nov. 30, 2022

Related U.S. Application Data
(60) Provisional application No. 63/175,492, filed on Apr. 15, 2021.
(60) Provisional application No. 63/108,173, filed on Oct. 30, 2020.
(60) Provisional application No. 63/034,289, filed on Jun. 3, 2020.

(51) Int. Cl. A61K 39/215; A61K 35/16; A61P 31/14

(52) U.S. Cl. CPC A61K 39/215; A61K 35/16; A61P 31/14; A61K 2039/505

(57) ABSTRACT

Hyperimmune IgG and/or IgM compositions and method for preparing thereof and method for obtaining hyperimmune human plasma from a donor. A liquid therapeutic hyperimmune globulin composition including human plasma-derived immunoglobulin G (IgG) having antibody titre between 250 and 2,500 per mg/mL of IgG and/or a SARS-CoV-2 neutralization activity (IC50 neutralization titer) between 1.5 and 15 per mg/mL of IgG for use in the treatment of coronavirus disease 2019 (COVID-19) in a patient in need thereof. A liquid therapeutic or prophylactic hyperimmune immunoglobulin composition including human plasma-derived immunoglobulin M (IgM) having a SARS-CoV-2 titre between 2,000 and 17,000 and/or a SARS-CoV-2 neutralization activity (IC50 neutralization titre) between 200 and 70,000, methods for preparing thereof, and the use thereof for the treatment or prophylaxis of COVID-19. A method for obtaining hyperimmune human plasma from a donor for use in the treatment of COVID-19.

The present invention is related to the field of pharmaceutical products. In particular, the present invention refers to liquid therapeutic hyperimmune globulin compositions comprising human plasma-derived immunoglobulin G (IgG) and/or immunoglobulin M (IgM) prepared from SARS-CoV-2 convalescent plasma obtained from patients that underwent coronavirus disease (COVID-19), methods for preparing thereof, and their use in the treatment of COVID-19 in a patient in need thereof. The present invention also refers to methods for obtaining hyperimmune human plasma from a donor for use in the treatment of coronavirus disease 2019 (COVID-19) in a patient in need thereof, wherein the donor has a laboratory confirmed diagnosis of COVID-19 and is in a convalescent noninfectious state.

COVID-19 is a respiratory tract infection caused by a newly emergent coronavirus, severe acute respiratory syndrome coronavirus 2 (SARS-CoV-2), that was first recognized in Wuhan, China, in December 2019 (WHO Interim guidance 13 Mar. 2020). Genetic sequencing of the virus suggests that SARS-CoV-2 is a betacoronavirus closely linked to the severe acute respiratory syndrome (SARS) virus (Team NCPERE 2020). While most people with COVID-19 develop mild or uncomplicated illness, approximately 14% develop severe disease requiring hospitalization and oxygen support and 5% require admission to an intensive care unit (Team NCPERE 2020). In severe cases, COVID-19 can be complicated by acute respiratory disease syndrome (ARDS), sepsis and septic shock, multiorgan failure, including acute kidney injury and cardiac injury (Yang et al., 2020). Older age and co-morbid disease have been reported as risk factors for death, and recent multivariable analysis confirmed older age, higher Sequential Organ Failure Assessment (SOFA) score and d-dimer >1 μg/L on admission were associated with higher mortality. That study also observed median duration of viral RNA detection was 20.0 days (interquartile range [IQR] 17.0-24.0) in survivors, but SARS-CoV-2 virus was detectable until death in non-survivors. The longest observed duration of viral shedding in survivors was 37 days (Huang et al., 2020; Zhou et al., 2020).

The lack of disease-directed therapeutic options for the treatment of COVID-19 has led to urgent interventions in anticipation of some potentially promising effects. Some antivirals are currently under evaluation. These include favipirivir (AVIGAN®) manufactured by Fuji film in Japan, Remdesivir manufactured by Gilead, and Kaletra® (lopinavir/ritonavir) commercially available for human immunodeficiency virus (HIV). There are also investigations of chloroquine and hydroxychloroquine as treatment modalities and potential applications for post-exposure prophylaxis according to Clinicaltrials.gov and other clinical trial registries. These and other potential therapeutic agents are described on the World Health Organization (WHO) website.

One of the approaches for the treatment of COVID-19 is the passive immunity; i.e. administering to a patient with plasma from donors that have been recovered from COVID-19 and have antibodies against this infection (hyperimmune plasma). This is known as SARS-CoV-2 convalescent human plasma, and it can be used in the treatment of COVID-19 in patients in need thereof to reduce all-cause mortality in requiring or not intensive care unit (ICU) admission patients and/or to reduce clinical severity, duration of hospital and ICU stay, dependency of oxygen and ventilator support.

Convalescent plasma has a long history of treatment of infectious diseases extending from the Spanish flu pandemic (Luke, T. C., et al., 2006) to more recent outbreaks of severe acute respiratory syndrome (SARS) (Soo, Y. O., et al., 2004), Middle East respiratory syndrome (MERS) (Ko, J. H., et al., 2018) and Ebola (Mupapa, K., et al., 1999).

There are, however some disadvantages of convalescent plasma, including that the nature, titer and neutralizing power of the antibodies therein can vary greatly from one donor to another. In addition, there are risks associated with the volume of convalescent plasma infused (transfusion-associated circulatory overload), the need to match donor/recipient blood types, the potential for transfusion-related allergic reactions and the lack of validated pathogen reduction processes.

The inventors of the present application have surprisingly discovered that the disadvantages of using anti-SARS-CoV-2 hyperimmune plasma from convalescent donors can be overcome through the purification and concentration of the specific antibodies into drug preparations. Thus, the present invention discloses hyperimmune globulin and/or immunoglobulin M (IgM) compositions made from pooled plasma of convalescent or vaccinated donors that can be used for the treatment of COVID-19 patients with severe clinical disease for reducing their symptoms, morbidity, and mortality.

In addition, the inventors of the present application have developed a method for preparing said hyperimmune globulin and/or IgM composition from SARS-CoV-2 convalescent human plasma resulting in a highly pure IgG and/or IgM composition with improved antibody titer and neutralization activity in relation to convalescent plasma.

Said method includes processing steps that have been validated for virus clearance and blood typing, reducing the risks of inadvertently transferring known infectious agents or triggering transfusion reactions. Thus, another advantage of the hyperimmune globulin and/or IgM composition over convalescent plasma is the pathogen clearance capability built into the processing. Both, convalescent plasma for transfusion and for manufacturing require the testing of common viral agents such as human immunodeficiency virus (HIV) and hepatitis B virus. However, in the event that novel viral contaminants are present, the method described herein to manufacture the hyperimmune globulin and/or IgM composition includes steps validated to remove or inactivate any viral pathogens.

The inventors of the present application have also surprisingly discovered that said hyperimmune plasma can be obtained from convalescent anti-SARS-CoV-2. The therapeutic use of convalescent plasma for COVID-19 is also interesting for patients with severe clinical disease for reducing their symptoms, morbidity, and mortality.

Passive administration of convalescent plasma can encompass typical risks associated with transfer of blood substances, which include inadvertent infection with another infectious disease agent and reactions to plasma constituents. With modern blood banking techniques that screen for blood-borne pathogens and match the blood type of donors and recipients, the risks of inadvertently transferring known infectious agents or triggering transfusion reactions are low.

### SUMMARY

The present invention discloses a liquid therapeutic hyperimmune globulin composition comprising human plasma-derived immunoglobulin G (IgG) with a purity of at least 97% of the total protein content and having a SARS-CoV-2 antibody titre between 250 and 2,500 per mg/mL of IgG and/or a SARS-CoV-2 neutralization activity between 1.5 and 15 per mg/mL of IgG.

In some embodiments, the purity of said human plasma-derived IgG is at least 98%, preferably at least 99% of the total protein content.

In some embodiments, the SARS-CoV-2 antibody titre is between 300 and 2,200 per mg/mL of IgG, preferably between 350 and 2,000 per mg/mL of IgG, more preferably between 400 and 1,900 per mg/mL of IgG, even more preferably between 450 and 1,800 per mg/mL of IgG, yet more preferably between 485 and 1,700 per mg/mL of IgG. In some embodiments the SARS-CoV-2 antibody titre is greater than 300, preferably greater than 500, preferably greater than 750, preferably greater than 1,000, preferably greater than 1,500, preferably greater than 2000 per mg/mL of IgG.

In some embodiments, the SARS-CoV-2 neutralization activity is between 1.8 and 12 per mg/mL of IgG, preferably between 2 and 10.5 per mg/mL of IgG, more preferably between 2.2 and 9.5 per mg/mL of IgG, yet more preferably between 2.4 and 8.8 per mg/mL of IgG. In some embodiments, the SARS-CoV-2 neutralization activity is greater than 1.5, preferably greater than 2, preferably greater than 2,5, preferably greater than 5, preferably greater than 7.5, preferably greater than 10, preferably greater than 12.5, per mg/mL of IgG.

In some embodiments, the human plasma-derived IgG content is between 5% and 20% (w/v). In more preferred embodiments, the human plasma-derived IgG content is between 9% and 11% (w/v).

In some embodiments, at least 90% of the human plasma-derived IgG is present as monomers and dimers.

In other embodiments, the content of immunoglobulin A (IgA) is equal or lower than 0.04 mg/ml and/or the content of immunoglobulin M (IgM) is equal or lower than 0.01 mg/ml.

The present invention also discloses a liquid therapeutic hyperimmune globulin composition comprising human plasma-derived immunoglobulin G (IgG) with a purity of at least 97% of the total protein content and having a SARS-CoV-2 antibody titre between 25,000 and 250,000 and/or a SARS-CoV-2 neutralization activity (IC50 neutralization titer) between 150 and 1,500.

In some embodiments, the purity of said human plasma-derived IgG is at least 98%, preferably at least 99% of the total protein content.

In some embodiments, the SARS-CoV-2 antibody titre is between 50,000 and 200,000, preferably between 75,000 and 150,000, more preferably between 100,000 and 125,000. In some embodiments the SARS-CoV-2 antibody titre is greater than 50,000, preferably greater than 75,000, preferably greater than 100,000, preferably greater than 150,000.

In some embodiments, the SARS-CoV-2 neutralization activity is between 200 and 1,250, preferably between 250 and 1,000, more preferably between 300 and 725, more preferably between 400 and 500.

In some embodiments, the SARS-CoV-2 neutralization activity is greater than 200, preferably greater than 300, preferably greater than 400, preferably greater than 500, preferably greater than 750, preferably greater than 1,000.

In some embodiments, the human plasma-derived IgG content is between 5% and 20% (w/v). In more preferred embodiments, the human plasma-derived IgG content is between 9% and 11% (w/v).

In some embodiments, at least 90% of the human plasma-derived IgG is present as monomers and dimers.

In other embodiments, the content of immunoglobulin A (IgA) is equal or lower than 0.04 mg/ml and/or the content of immunoglobulin M (IgM) is equal or lower than 0.01 mg/ml.

The present invention also discloses liquid therapeutic hyperimmune globulin compositions as described herein, for use in the treatment of coronavirus disease 2019 (COVID-19) in a patient in need thereof.

The present invention also discloses a method for preparing a liquid therapeutic hyperimmune globulin composition as described herein, from a starting solution comprising anti-SARS-CoV-2 IgG antibodies, the method comprising the sequential steps a) through e) of:
a) adjusting the pH of the starting solution to be within a range of from about 3.8 to about 4.5 to form an intermediate solution comprising dissolved antibodies,
b) adding a source of caprylate ions to the intermediate solution of step a) and adjusting the pH of the intermediate solution to be within a range of from about 5.0 to about 5.2 to form a precipitate and a supernatant solution comprising dissolved antibodies,
c) incubating the supernatant solution under conditions of time, temperature and caprylate ion concentration to inactivate substantially all viruses,
d) contacting the supernatant solution with at least one ion exchange resin under conditions that allow binding of at least some of the other substances including IgA or IgM to the resin while not allowing binding of the antibodies including IgG to the resin, and
e) collecting the IgG antibodies,
   wherein the starting solution is SARS-CoV-2 convalescent human plasma.

In some embodiments, said SARS-CoV-2 convalescent human plasma is a pool of plasma samples from at least two convalescent donors. In other embodiments, said

SARS-CoV-2 convalescent human plasma is tested negative for at least one of blood-borne pathogens and human leukocyte antigen (HLA) antibody. In other embodiments, said SARS-CoV-2 convalescent human plasma is tested for blood type.

The present invention also discloses a liquid therapeutic hyperimmune IgM composition comprising human plasma-derived immunoglobulin M (IgM) with a purity of at least 85% of the total immunoglobulin content and having a SAR-CoV-2 titre between 2,000 and 17,000 and/or a SARS-CoV-2 neutralization activity (IC50 neutralization titre) between 200 and 70,000.

In some embodiments, the purity of said human plasma-derived IgM is at least 90%, preferably at least 94%, more preferably at least 95% or at least 96% of the total immunoglobulin content.

In some embodiments, the SARS-CoV-2 antibody titre is between 3,000 and 13,000, preferably between 4,000 and 12,000, more preferably between 5,000 and 11,000. In some embodiments the SARS-CoV-2 antibody titre is greater than 2,000, preferably greater than 4,000, preferably greater than 5,000, preferably greater than 6,000.

In some embodiments, the SARS-CoV-2 neutralization activity is between 300 and 60,000, preferably between 500 and 50,000, more preferably between 1,000 and 40,000, more preferably between 2,000 and 30,000.

In some embodiments, the SARS-CoV-2 neutralization activity is greater than 200, preferably greater than 300, preferably greater than 400, preferably greater than 500, preferably greater than 800, preferably greater than 1,000, preferably greater than 2,000, preferably, greater than 5,000, preferably greater than 8,000, preferably greater than 10,000.

In some embodiments, the human plasma-derived IgM content is between 1% and 10% (w/v). In more preferred embodiments, the human plasma-derived IgM content is between 1.5% and 5% (w/v). In more preferred embodiments, the human plasma-derived IgM content is around 2.5% (w/v). In some embodiments, the human plasma-derived IgM content is greater than 1% (w/v), or greater than 2% (w/v), or greater than 3% (w/v), or greater than 4% (w/v), or greater than 5% (w/v).

In some embodiments, at least 75% of the human plasma-derived IgM is present as pentamers. In more preferred embodiments, at least 90% of the human plasma-derived IgM is present as pentamers. In more preferred embodiments, at least 94% is present as pentamers. In more preferred embodiments, at least 95% is present or at least 98% is present as pentamers.

In other embodiments, the content of immunoglobulin G (IgG) is equal or lower than 7% of the total immunoglobulin, preferably equal or lower than 2% and/or the content of immunoglobulin A (IgA) is equal or lower than 7% of the total immunoglobulin, preferably equal or lower than 4%.

The present invention also refers to liquid therapeutic hyperimmune IgM compositions as described herein, for use in the treatment of coronavirus disease 2019 (COVID-19) in a patient in need thereof.

The present invention also refers to a method for preparing a liquid therapeutic hyperimmune IgM composition as described herein, from a starting solution comprising anti-SARS-CoV-2 IgM antibodies, the method comprising the sequential steps a) through f) of:
a) precipitation of said IgM using polyethylene glycol (PEG);
b) resuspension of the precipitated IgM;
c) adsorption chromatography;
d) isoagglutinin affinity chromatography;
e) nanofiltration; and
f) ultrafiltration/diafiltration.
   wherein the starting solution is obtained from SARS-CoV-2 convalescent human plasma.

In the process of the present invention, the starting material used can come from different sources. For example, the source material for the described IgM process can be column strip from either of the two Gamunex process (as described in U.S. Pat. No. 6,307,028) anion-exchange chromatography columns (Q sepharose or ANX sepharose) operated in series. In that process, IgG is purified from Fraction II+III paste generated from the Grifols plasma fractionation processes, as described in the mentioned patent. Briefly, after collecting IgG in the anion exchange columns flow through, bound protein, almost exclusively immunoglobulin (IgM, IgG and IgA), is eluted by applying a buffer comprising 0.5 M sodium acetate at pH 5.2. Columns are stripped separately wherein either or both fractions can be further processed to purify IgM. The abundance ratios of each of the three immunoglobulins differ significantly between the two column strips.

In some embodiments, said SARS-CoV-2 convalescent human plasma is a pool of plasma samples from at least two convalescent donors. In other embodiments, said SARS-CoV-2 convalescent human plasma is tested negative for at least one of blood-borne pathogens and human leukocyte antigen (HLA) antibody. In other embodiments, said SARS-CoV-2 convalescent human plasma is tested for blood type.

In one embodiment, said precipitation step a) is performed at a pH between 4.5 and 6.5.

In one embodiment, said PEG is at a concentration between 5% (w/v) and 11% (w/v). Preferably, said PEG is PEG-3350.

In one embodiment, said precipitation step is carried out for no less than 30 min.

In one embodiment, said absorption chromatography is ceramic hydroxyapatite (CHT) chromatography.

In one embodiment, the loading or washing solution of the ceramic hydroxyapatite (CHT) comprises NaCl, preferably at a concentration between 0.5 M and 2.0 M.

In one embodiment, the washing solution of the ceramic hydroxyapatite (CHT) comprises urea, preferably at a concentration between 1 M and 5 M.

In one embodiment, said step d) of removing isoagglutinins A/B is performed by affinity chromatography using A/B trisaccharides as ligand.

In one embodiment, said step d) of removing isoagglutinins A/B is performed using at least two affinity columns in series, at least one with trisaccharide A as a ligand, and at least one with trisaccharide B as a ligand or step d) is performed using at least one affinity column containing a mixture with trisaccharide A and trisaccharide B as a ligand.

In one embodiment, said nanofiltration step e) is performed through a filter of 35 nm or greater of average pore size.

In one embodiment, said nanofiltration step e) is performed using a buffer comprising at least 0.5 M of Arginine-HCl at a pH between 6.0 and 9.0. Preferably, said nanofiltration step e) is performed using a buffer comprising at least 0.5 M of Arginine-HCl at a pH between 7.0 and 8.0.

In one embodiment, said ultrafiltration step (f) is performed at a pH between 4.5 and 5.0.

In one embodiment, said diafiltration step e) is performed with a succinate buffer or a buffer containing amino acids at a pH between 3.8 and 4.8.

In one embodiment, said amino acids are glycine, alanine, proline, valine, or hydroxyproline or a mixture thereof.

The present invention refers to a method for obtaining hyperimmune human plasma from a donor for use in the treatment of coronavirus disease 2019 (COVID-19), wherein said donor has a laboratory confirmed diagnosis of COVID-19 and said donor is in a convalescent noninfectious state.

In some embodiments, said donor is symptomatic or asymptomatic for COVID-19.

In some embodiments, symptoms of COVID-19 are one or more of fever, tiredness, dry cough, ache, pain, nasal congestion, runny nose, sore throat, or diarrhea.

In some embodiments, said donor is tested positive or negative for COVID-19 as determined by any nucleic acid technology (NAT) test and/or by any serology test for detecting antibodies anti-SARS-CoV-2.

In some embodiments, if said donor is asymptomatic and is tested positive for antibodies anti-SARS-CoV-2 but negative in the NAT test, is immediately eligible for plasma donation.

In some embodiments, if said donor is asymptomatic and is only tested positive for the NAT test, or is positive for the NAT test with a subsequent positive for antibodies anti-SARS-CoV-2, said donor is eligible for plasma donation 28 days after collecting the sample for the NAT test.

In some embodiments, if said donor is asymptomatic and is only positive for anti-SARS-CoV-2 antibodies, said donor is eligible for plasma donation after 7 days after the serology test.

In some embodiments, if said donor is asymptomatic and is positive for the NAT test with a subsequent negative for the NAT test, said donor is eligible for plasma donation 14 days after the negative NAT test.

In some embodiments, if said donor is symptomatic and is only positive for the NAT test, or is positive for the NAT test with a subsequent positive for anti-SARS-COV-2 antibodies, or is only positive for anti-SARS-COV-2 antibodies, or is positive for the NAT test with a subsequent negative for anti-SARS-COV-2 antibodies, said donor is eligible for plasma donation 28 days after cessation of all symptoms.

In some embodiments, if said donor is symptomatic and is positive for the NAT test with a subsequent positive for the NAT test, said donor is eligible for plasma donation 28 days after cessation of all symptoms or 28 days after the second NAT test, whichever is later.

In some embodiments, if said donor is symptomatic and is positive for the NAT test with a subsequent negative for the NAT test, said donor is eligible for plasma donation 14 days after cessation of all symptoms.

In some embodiments, said plasma is screened at least for blood-borne pathogens and blood type.

In some embodiments, said plasma is freezed after collection.

### DETAILED DESCRIPTION

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

As used in this specification and claims, the singular forms “a,” “an” and “the” include plural references unless the content clearly dictates otherwise.

As used herein, “about” means a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “Nucleic acid technology or NAT”, as used herein, refers to any amplification-based or transcription-based method for detection and quantitation of a target nucleic acid. Numerous amplification-based methods are well known and established in the art, such as PCR, its variation RT-PCR, strand displacement amplification (SDA), thermophilic SDA (tSDA), rolling circle amplification (RCA), helicase dependent amplification (HDA), or loop-mediated isothermal amplification (LAMP). Transcription-based amplification methods commonly used in the art include nucleic acid sequence based amplification (NASBA), Qβ replicase, self-sustained sequence replication or transcription-mediated amplification (TMA).

The term “convalescent plasma”, as used herein, refers to plasma collected from previously infected individuals. Thus, the term “convalescent anti-SARS-CoV-2 plasma” or “SARS-CoV-2 convalescent human plasma” as used herein refer to convalescent plasma collected from individuals previously infected with SARS-CoV-2 that have recovered from COVID-19 and that are in a convalescent noninfectious state.

The term “hyperimmune”, as used herein, refers to products or compositions comprising an elevated level of antibodies, e.g, polyclonal antibodies, to one or more specific antigens, which is obtained from plasma and/or serum.

The term “plasma-derived”, as used herein, refers to products that are made from donated human blood, from which the plasma or plasma proteins (such as immonolubulins) are separated or removed and made into proteins concentrates or fresh frozen plasma. Plasma-derived products can be made from pools of samples from multiple donors, usually from no less than 1,000 donors.

The terms “neutralization activity” or “IC50 neutralization titre” as used herein, are interchangeable and refer to the amount of the liquid therapeutic hyperimmune globulin composition of the present invention, required for neutralizing or inhibiting 50% of infection by SARS-CoV-2.

Although this disclosure is in the context of certain embodiments and examples, those skilled in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of skill in the art based upon this disclosure.

It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

It should be understood, however, that this description, while indicating preferred embodiments of the disclosure, is given by way of illustration only, since various changes and modifications within the spirit and scope of the disclosure will become apparent to those skilled in the art.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the embodiments herein described.

A first aspect of the present invention relates to a liquid therapeutic hyperimmune globulin composition comprising human plasma-derived immunoglobulin G (IgG) with a purity of at least 97% of the total protein content.

In some embodiments, said composition comprises human plasma-derived immunoglobulin G (IgG) with a purity of at least 98%, at least 99%, at least 99.5%, at least 99.8% or at least 99.9% of the total protein content. In some embodiments said composition comprises human plasma-derived immunoglobulin G (IgG) with a purity of about 100%.

The liquid therapeutic hyperimmune globulin composition comprising human plasma-derived immunoglobulin G (IgG) of the present invention can have a SARS-CoV-2 antibody titre between 25,000 and 250,000. In some embodiments, the SARS-CoV-2 antibody titre of the composition of the present invention is between 50,000 and 200,000. In other embodiments, said SARS-CoV-2 antibody titre is between 75,000 and 150,000. In other embodiments, said SARS-CoV-2 antibody titre is between 100,000 and 125,000. In other embodiments, said SARS-CoV-2 antibody titre is between 110,000 and 120,000. In some embodiments the SARS-CoV-2 antibody titre is greater than 50,000, preferably greater than 75,000, preferably greater than 100,000, preferably greater than 125,000, preferably greater than 150,000, preferably greater than 200,000.

In some embodiments of the present invention, the SARS-CoV-2 antibody titre of the liquid therapeutic hyperimmune globulin composition is increased by at least 2-fold with respect to the SARS-CoV-2 antibody titre in the pooled plasma from which said composition is prepared. In other embodiments, said SARS-CoV-2 antibody titre is increased by at least 5-fold, preferably by at least 10-fold, preferably by at least 15-fold, preferably by at least 25-fold, preferably by at least 30-fold, with respect to the SARS-CoV-2 antibody titre in the pooled plasma from which said composition is prepared.

The SARS-CoV-2 antibody titre of the present composition can be determined using, for example, the human Anti-SARS-CoV-2 Virus Spike 1 (S1) IgG assay from Alpha Diagnostics Ltd. (Switzerland). However, other assays known by the skilled person can also be used.

The SARS-CoV-2 antibody titre of the liquid therapeutic hyperimmune globulin composition comprising human plasma-derived immunoglobulin G (IgG) of the present invention can also be normalized per mg/ml of IgG. Thus, in some embodiments the SARS-CoV-2 antibody titre of the composition of the present invention is between 250 and 2,500 per mg/mL of IgG. In other embodiments, said SARS-CoV-2 antibody titre is between 300 and 2,200 per mg/mL of IgG. In other embodiments, said SARS-CoV-2 antibody titre is between 350 and 2,000 per mg/mL of IgG. In other embodiments, said SARS-CoV-2 antibody titre is between 400 and 1,900 per mg/mL of IgG. In other embodiments, said SARS-CoV-2 antibody titre is between 450 and 1,800 per mg/mL of IgG. In other embodiments, said SARS-CoV-2 antibody titre is between 485 and 1,700 per mg/mL of IgG. In some embodiments, said SARS-CoV-2 antibody titre is greater than 300, preferably greater than 500, preferably greater than 750, preferably greater than 1,000, preferably greater than 1,500, preferably greater than 2000 per mg/mL of IgG.

The liquid therapeutic hyperimmune globulin composition comprising human plasma-derived immunoglobulin G (IgG) of the present invention can have a SARS-CoV-2 neutralization activity (IC50 neutralization titer) between 150 and 1,500.In some embodiments, the SARS-CoV-2 neutralization activity is between 200 and 1,250. In more preferred embodiments, said neutralization activity is between 250 and 1,000. In yet more preferred embodiments, said neutralization activity is between 300 and 725.

In more preferred embodiments, said neutralization activity is between 400 and 500. In some embodiments, the SARS-CoV-2 neutralization activity is greater than 150, preferably greater than 200, preferably greater than 300, preferably greater than 400, preferably greater than 500, preferably greater than 750, preferably greater than 1,000.

In some embodiments of the present invention, the SARS-CoV-2 neutralization activity of the liquid therapeutic hyperimmune globulin composition is increased by at least 2-fold with respect to the SARS-CoV-2 neutralization activity in the pooled plasma from which said composition is prepared. In other embodiments, said SARS-CoV-2 neutralization activity is increased by at least 5-fold, preferably by at least 7-fold, preferably by at least 10-fold, preferably by at least 13-fold, with respect to the SARS-CoV-2 neutralization activity in the pooled plasma from which said composition is prepared.

The SARS-CoV-2 neutralization activity (IC50 neutralization titre) of the present composition can be determined using, for example, an immunofluorescence-based neutralization assay in which inhibition of infection of cultured eukaryotic cells, such as Vero (CCL-81) cells by SARS-CoV-2, is tested. However, the skilled person knows other assays that can be used to determine the SARS-CoV-2 neutralization activity (IC50) of the present composition.

The SARS-CoV-2 neutralization activity (IC50 neutralization titer) of the liquid therapeutic hyperimmune globulin composition comprising human plasma-derived immunoglobulin G (IgG) of the present invention can also be normalized per mg/ml of IgG. Thus, in some embodiments the SARS-CoV-2 neutralization activity of the composition of the present invention is between 1.5 and 15 per mg/mL of IgG. In other embodiments, said SARS-CoV-2 neutralization activity is between 1.8 and 12 per mg/mL of IgG. In more preferred embodiments, said SARS-CoV-2 neutralization activity is between 2 and 10.5 per mg/mL of IgG. In yet more preferred embodiments, said SARS-CoV-2 neutralization activity is between 2.2 and 9.5 per mg/mL of IgG. In even more preferred embodiments, said SARS-CoV-2 neutralization activity is between 2.4 and 8.8 per mg/mL of IgG. In some embodiments, the SARS-CoV-2 neutralization activity is greater than 1.5, preferably greater than 2, preferably greater than 2.5, preferably greater than 5, preferably greater than 7.5, preferably greater than 10, preferably greater than 12.5, per mg/mL of IgG.

The liquid therapeutic hyperimmune globulin composition comprising human plasma-derived immunoglobulin G (IgG) of the present invention can be defined by any of the above antibody titre and/or neutralization activity.

In some embodiments, the human plasma-derived IgG content of the liquid therapeutic hyperimmune globulin composition of the present invention is between 5% and 20% (w/v). In more preferred embodiments, the human plasma-derived IgG content is between 7% and 15% (w/v). In more preferred embodiments, the human plasma-derived IgG content is between 9% and 11% (w/v). In more preferred embodiments, the human plasma-derived IgG content is around 10% (w/v).

In some embodiments, at least 90% of the human plasma-derived IgG of the liquid therapeutic hyperimmune globulin composition of the present invention is present as monomers and dimers. In more preferred embodiments, at least 95% of the human plasma-derived IgG is present as monomers and dimers. In more preferred embodiments, at least 98% of the human plasma-derived IgG is present as monomers and dimers. In more preferred embodiments, at least 99% of the human plasma-derived IgG is present as monomers and dimers. In more preferred embodiments, at least 99.8% of the human plasma-derived IgG is present as monomers and dimers.

The liquid therapeutic hyperimmune globulin composition of the present invention is a highly purified IgG composition, but it may comprise residual amounts of other immunoglobulins, such as immunoglobulin A (IgA) or immunoglobulin M (IgM). In some embodiments, the content of IgA in said composition is equal or lower than 0.04 mg/ml. In more preferred embodiments, the content of IgA in said composition is equal or lower than 0.038 mg/ml. In some embodiments, the content of IgM in said composition is equal or lower than 0.01 mg/ml.

The liquid therapeutic hyperimmune globulin composition of the present invention can be used in the treatment of coronavirus disease 2019 (COVID-19) in a patient in need thereof. The skilled person knows the preferred dosage regimes and administration route (intravenous route is preferred for immunoglobulin compositions) for the treatment of COVID-19 with the hyperimmune globulin composition of the present invention.

A second aspect of the present invention relates to a method for preparing the liquid therapeutic hyperimmune globulin composition as described herein, from a starting solution comprising anti-SARS-CoV-2 IgG antibodies and comprising the sequential steps a) through e) of
a) adjusting the pH of the starting solution to be within a range of from about 3.8 to about 4.5 to form an intermediate solution comprising dissolved antibodies,
b) adding a source of caprylate ions to the intermediate solution of step a) and adjusting the pH of the intermediate solution to be within a range of from about 5.0 to about 5.2 to form a precipitate and a supernatant solution comprising dissolved antibodies,
c) incubating the supernatant solution under conditions of time, temperature and caprylate ion concentration to inactivate substantially all viruses,
d) contacting the supernatant solution with at least one ion exchange resin under conditions that allow binding of at least some of the other substances including IgA or IgM to the resin while not allowing binding of the antibodies including IgG to the resin, and
e) collecting the IgG antibodies,
   wherein the starting solution is SARS-CoV-2 convalescent human plasma.

In some preferred embodiments, the method for preparing the liquid therapeutic hyperimmune globulin composition of the present invention further comprises a non-sequential step f) of eluting IgA or IgM from the ion exchange resin column.

In other embodiments, the method for preparing the liquid therapeutic hyperimmune globulin composition as described herein is as disclosed in U.S. Pat No. 6,307,028, which is incorporated by reference herein.

In some embodiments of the method for preparing the liquid therapeutic hyperimmune globulin composition as described herein, the SARS-CoV-2 convalescent human plasma used as starting solution is a pool of plasma samples from a plurality of convalescent donors. In more preferred embodiments, the SARS-CoV-2 convalescent human plasma used as starting solution is a pool of plasma samples from at least two convalescent donors, preferably from at least 50 convalescent donors, more preferably from at least 100 convalescent donors, yet more preferably from at least 50 convalescent donors, even more preferably from at least 100 convalescent donors.

In some embodiments, said SARS-CoV-2 convalescent human plasma is tested negative for at least one of blood-borne pathogens and human leukocyte antigen (HLA) antibody. Any known method for testing blood-borne pathogens can be used. Similarly, any known method for testing the presence of human leukocyte antigen (HLA) antibodies can be used.

In other embodiments, said SARS-CoV-2 convalescent human plasma is tested for blood type.

A further aspect of the present invention relates to methods for obtaining the SARS-CoV-2 convalescent human plasma that is used as starting solution in the method for preparing the liquid therapeutic hyperimmune globulin composition as described herein.

In some embodiments, SARS-CoV-2 convalescent human plasma is obtained following any method known by the skilled person. In other embodiments, SARS-CoV-2 convalescent human plasma is obtained following the method as disclosed in U.S. 63/034,289 (incorporated by reference herein).

Thus, in some embodiments, the method for obtaining SARS-CoV-2 convalescent human comprises selecting at least a donor that has a laboratory confirmed diagnosis of COVID-19 and is in a convalescent noninfectious state. In some embodiments, said donor is symptomatic or asymptomatic for COVID-19. In some embodiments, symptoms of COVID-19 are one or more of fever, tiredness, dry cough, ache, pain, nasal congestion, runny nose, sore throat, or diarrhea.

In some embodiments, said donor is tested positive or negative for COVID-19 as determined by any nucleic acid technology (NAT) test and/or by any serology test for detecting antibodies anti-SARS-CoV-2 and/or by any antigen test for detecting SARS-CoV-2 antigens.

In some embodiments, if said donor is asymptomatic and is tested positive for antibodies anti-SARS-CoV-2 but negative in the NAT test, is immediately eligible for plasma donation.

In some embodiments, if said donor is asymptomatic and is only tested positive for the NAT test or the antigen test, or is positive for the NAT test with a subsequent positive for antibodies anti-SARS-CoV-2, said donor is eligible for plasma donation 28 days after collecting the sample for the NAT test.

In some embodiments, if said donor is asymptomatic and is only positive for anti-SARS-CoV-2 antibodies, said donor is eligible for plasma donation after 7 days after the serology test.

In some embodiments, if said donor is asymptomatic and is positive for the NAT test or antigen test with a subsequent negative for the NAT test, said donor is eligible for plasma donation 14 days after the negative NAT test.

In some embodiments, if said donor is symptomatic and is only positive for the NAT test, or is positive for the NAT test with a subsequent positive for anti-SARS-CoV-2 antibodies, or is only positive for anti-SARS-CoV-2 antibodies, or is positive for the NAT test with a subsequent negative for anti-SARS-CoV-2 antibodies, said donor is eligible for plasma donation 28 days after cessation of all symptoms.

In some embodiments, if said donor is symptomatic and is positive for the NAT test with a subsequent positive for the NAT test, said donor is eligible for plasma donation 28 days after cessation of all symptoms or 28 days after the second NAT test, whichever is later.

In some embodiments, if said donor is symptomatic and is positive for the NAT test with a subsequent negative for the NAT test, said donor is eligible for plasma donation 14 days after cessation of all symptoms.

In some embodiments, said plasma is screened at least for blood-borne pathogens and blood type. In other embodiments, said plasma is tested negative for human leukocyte antigen (HLA) antibody.

In some embodiments, said plasma is freezed after collection. In some embodiments, said plasma is collected by plasmapheresis.

In some embodiments, the method for obtaining SARS-CoV-2 convalescent human comprises treatment of said plasma with methylene blue.

A third aspect of the present invention relates to a liquid therapeutic hyperimmune globulin composition comprising human plasma-derived immunoglobulin M (IgM) with a purity of at least 85% of the total immunoglobulin content and having a SARS-CoV-2 titre between 2,000 and 17,000 and/or a SARS CoV 2 neutralization activity (IC50 neutralization titre) between 200 and 70,000.

In some embodiments, said composition comprises human plasma-derived immunoglobulin M (IgM) with a purity of at least 90%, at least 94%, at least 95%, at least 96%, of the total immunoglobulin content.

In some embodiments, the SARS-CoV-2 antibody titre is between 3,000 and 15,000, preferably between 4,000 and 12,000, more preferably between 5,000 and 11,000. In some embodiments the SARS-CoV-2 antibody titre is greater than 2,000, preferably greater than 4,000, preferably greater than 5,000, preferably greater than 6,000.

In some embodiments, the SARS-CoV-2 neutralization activity is between 300 and 60,000, preferably between 500 and 50,000, more preferably between 1,000 and 40,000, more preferably between 2,000 and 30,000.

In some embodiments, the SARS-CoV-2 neutralization activity is greater than 200, preferably greater than 300, preferably greater than 400, preferably greater than 500, preferably greater than 800, preferably greater than 1,000, preferably greater than 2,000, preferably, greater than 5,000, preferably greater than 8,000, preferably greater than 10,000.

In some embodiments of the present invention, the SARS-CoV-2 neutralization activity of the liquid therapeutic hyperimmune globulin composition is increased by at least 2-fold with respect to the SARS CoV-2 neutralization activity in the pooled plasma from which said composition is prepared. In other embodiments, said SARS-CoV-2 neutralization activity is increased by at least 5-fold, preferably by at least 7-fold, preferably by at least 10-fold, preferably by at least 15-fold, preferably by at least 20-fold, more preferably at least 25-fold, more preferably at least 30-fold with respect to the SARS-CoV-2 neutralization activity in the pooled plasma from which said composition is prepared.

The SARS-CoV-2 neutralization activity (IC50 neutralization titre) of the present composition can be determined using, for example, an immunofluorescence-based neutralization assay in which inhibition of infection of cultured eukaryotic cells, such as Vero (CCL-81) cells by SARS-CoV-2, is tested. However, the skilled person knows other assays that can be used to determine the SARS-CoV-2 neutralization activity (IC50) of the present composition. For example, the Cytopathic-Cytotoxicity Luminometry Assay (COLA), Plaque Forming Units (PFU), or Median Tissue Culture Infectious Dose (TCID50), among others.

The SARS-CoV-2 neutralization activity (IC50 neutralization titre) of the liquid therapeutic hyperimmune globulin composition comprising human plasma-derived immunoglobulin M (IgM) of the present invention can also be normalized per mg/ml of IgM. Thus, in some embodiments the SARS-CoV-2 neutralization activity of the composition of the present invention is between 1.5 and 15 per mg/mL of IgM. In other embodiments, said SARS-CoV-2 neutralization activity is between 1.8 and 12 per mg/mL of IgM. In more preferred embodiments, said SARS-CoV-2 neutralization activity is between 2 and 10.5 per mg/mL of IgM. In yet more preferred embodiments, said SARS-CoV-2 neutralization activity is between 2.2 and 9.5 per mg/mL of IgM. In even more preferred embodiments, said SARS-CoV-2 neutralization activity is between 2.4 and 8.8 per mg/mL of IgM. In some embodiments, the SARS-CoV-2 neutralization activity is greater than 1.5, preferably greater than 2, preferably greater than 2.5, preferably greater than 5, preferably greater than 7.5, preferably greater than 10, preferably greater than 12.5, per mg/mL of IgM.

In some embodiments, the human plasma-derived IgM content of the liquid therapeutic hyperimmune globulin composition of the present invention is between 1% and 10% (w/v). In more preferred embodiments, the human plasma-derived IgM content is between 1.5% and 5% (w/v). In more preferred embodiments, the human plasma-derived IgM content is around 2.5% (w/v).

In some embodiments, at least 75% of the human plasma-derived IgM of the liquid therapeutic hyperimmune globulin composition of the present invention is present as pentamers. In more preferred embodiments, at least 90% of the human plasma-derived IgM is present as pentamers. In more preferred embodiments, at least 94% of the human plasma-derived IgM is present pentamers. In more preferred embodiments, at least 95% of the human plasma-derived IgM is present as pentamers. In more preferred embodiments, at least 96% of the human plasma-derived IgM is present as pentamers.

The liquid therapeutic hyperimmune globulin composition of the present invention is a highly purified IgM composition, but it may comprise residual amounts of other immunoglobulins, such as immunoglobulin A (IgA) or immunoglobulin G (IgG). In some embodiments, the content of IgA in said composition is equal or lower than 7% of the total immunoglobulin content. In more preferred embodiments, the content of IgA in said composition is equal or lower than 4%. In some embodiments, the content of IgG in said composition is equal or lower than 7% of the total immunoglobulin content. In more preferred embodiments, the content of IgG in said composition is equal or lower than 2%.

The liquid therapeutic hyperimmune globulin composition comprising plasma-derived IgM of the present invention can be used in the treatment of coronavirus disease 2019 (COVID-19) in a patient in need thereof. The skilled person knows the preferred dosage regimes and administration route (intravenous route is preferred for immunoglobulin compositions) for the treatment of COVID-19 with the hyperimmune globulin composition of the present invention.

A fourth aspect of the present invention relates to a method for preparing the liquid therapeutic hyperimmune globulin composition as described herein, from a starting solution comprising anti SARS CoV-2 IgM antibodies and comprising the sequential steps a) through f) of:
a) precipitation of said IgM using polyethylene glycol (PEG);
b) resuspension of the precipitated IgM;
c) adsorption chromatography;
d) isoagglutinin affinity chromatography;
e) nanofiltration; and
f) ultrafiltration/diafiltration.
   wherein the starting solution is obtained from SARS-CoV-2 convalescent human plasma.

In the process of the present invention, the starting material used can come from different sources. For example, the source material for the described IgM process can be column strip from either of the two Gamunex process (as described in U.S. Pat. No. 6,307,028) anion-exchange chromatography columns (Q sepharose or ANX sepharose) operated in series. In that process, IgG is purified from Fraction II+III paste generated from the Grifols plasma fractionation processes, as described in the mentioned patent. Briefly, after collecting IgG in the anion exchange columns flow through, bound protein, almost exclusively immunoglobulin (IgM, IgG and IgA), is eluted by applying a buffer comprising 0.5 M sodium acetate at pH 5.2. Columns are stripped separately wherein either or both fractions can be further processed to purify IgM. The abundance ratios of each of the three immunoglobulins differ significantly between the two column strips.

In some embodiments of the method for preparing the liquid therapeutic hyperimmune IgM composition as described herein, the SARS-CoV-2 convalescent human plasma used as starting solution is a pool of plasma samples from a plurality of convalescent donors. In more preferred embodiments, the SARS CoV-2 convalescent human plasma used as starting solution is a pool of plasma samples from at least two convalescent donors, preferably from at least 50 convalescent donors, more preferably from at least 100 convalescent donors, yet more preferably from at least 50 convalescent donors, even more preferably from at least 100 convalescent donors.

In some embodiments, said SARS-CoV-2 convalescent human plasma is tested negative for at least one of blood-borne pathogens and human leukocyte antigen (HLA) antibody. Any known method for testing blood-borne pathogens can be used. Similarly, any known method for testing the presence of human leukocyte antigen (HLA) antibodies can be used.

In other embodiments, said SARS-CoV-2 convalescent human plasma is tested for blood type.

A further aspect of the present invention relates to methods for obtaining the SARS-CoV-2 convalescent human plasma that is used as starting solution in the method for preparing the liquid therapeutic hyperimmune globulin composition as described herein.

In some embodiments, SARS-CoV-2 convalescent human plasma is obtained following any method known by the skilled person. In other embodiments, SARS-CoV-2 convalescent human plasma is obtained following the method as disclosed in U.S. 63/034,289, as will be explained below.

Thus, in some embodiments, the method for obtaining SARS-CoV-2 convalescent human comprises selecting at least a donor that has a laboratory confirmed diagnosis of COVID-19 and is in a convalescent noninfectious state. In some embodiments, said donor is symptomatic or asymptomatic for COVID-19. In some embodiments, symptoms of COVID-19 are one or more of fever, tiredness, dry cough, ache, pain, nasal congestion, runny nose, sore throat, or diarrhea.

In some embodiments, said donor is tested positive or negative for COVID-19 as determined by any nucleic acid technology (NAT) test and/or by any serology test for detecting antibodies anti-SARS-CoV-2 and/or by any antigen test for detecting SARS-CoV-2 antigens.

In some embodiments, if said donor is asymptomatic and is tested positive for antibodies anti SARS CoV-2 but negative in the NAT test, is immediately eligible for plasma donation.

In some embodiments, if said donor is asymptomatic and is only tested positive for the NAT test or the antigen test, or is positive for the NAT test with a subsequent positive for antibodies anti-SARS-CoV-2, said donor is eligible for plasma donation 28 days after collecting the sample for the NAT test.

In some embodiments, if said donor is asymptomatic and is only positive for anti-SARS-CoV-2 antibodies, said donor is eligible for plasma donation after 7 days after the serology test.

In some embodiments, if said donor is asymptomatic and is positive for the NAT test or antigen test with a subsequent negative for the NAT test, said donor is eligible for plasma donation 14 days after the negative NAT test.

In some embodiments, if said donor is symptomatic and is only positive for the NAT test, or is positive for the NAT test with a subsequent positive for anti-SARS-CoV-2 antibodies, or is only positive for anti SARS-CoV-2 antibodies, or is positive for the NAT test with a subsequent negative for anti SARS CoV-2 antibodies, said donor is eligible for plasma donation 28 days after cessation of all symptoms.

In some embodiments, if said donor is symptomatic and is positive for the NAT test with a subsequent positive for the NAT test, said donor is eligible for plasma donation 28 days after cessation of all symptoms or 28 days after the second NAT test, whichever is later.

In some embodiments, if said donor is symptomatic and is positive for the NAT test with a subsequent negative for the NAT test, said donor is eligible for plasma donation 14 days after cessation of all symptoms.

In some embodiments, said plasma is screened at least for blood-borne pathogens and blood type. In other embodiments, said plasma is tested negative for human leukocyte antigen (HLA) antibody.

In some embodiments, said plasma is frozen after collection. In some embodiments, said plasma is collected by plasmapheresis.

In some embodiments, the method for obtaining SARS-CoV-2 convalescent human plasma comprises treatment of said plasma with methylene blue.

In the process of the present invention, the starting material used can come from different sources. For example, the source material for the described IgM process can be column strip from either of the two Gamunex process (as described in U.S. Pat. No. 6,307,028) anion-exchange chromatography columns (Q sepharose or ANX sepharose) operated in series. In that process, IgG is purified from Fraction II+III paste generated from the Grifols plasma fractionation processes, as described in the mentioned patent. Briefly, after collecting IgG in the anion exchange columns flow through, bound protein, almost exclusively immunoglobulin (IgM, IgG and IgA), is eluted by applying a buffer comprising 0.5 M sodium acetate at pH 5.2. Columns are stripped separately wherein either or both fractions can be further processed to purify IgM. The abundance ratios of each of the three immunoglobulins differ significantly between the two column strips.

A fifth aspect of the present invention relates to a method for obtaining hyperimmune human plasma from a donor for use in the treatment of coronavirus disease 2019 (COVID-19), wherein said donor has a laboratory confirmed diagnosis of COVID-19 and said donor is in a convalescent noninfectious state.

In some embodiments, said donor is symptomatic or asymptomatic for COVID-19.

In some embodiments, symptoms of COVID-19 are one or more of fever, tiredness, dry cough, ache, pain, nasal congestion, runny nose, sore throat, or diarrhea.

In some embodiments, said donor is tested positive or negative for COVID-19 as determined by any nucleic acid technology (NAT) test and/or by any serology test for detecting antibodies anti-SARS-CoV-2.

In some embodiments, if said donor is asymptomatic and is tested positive for antibodies anti-SARS-CoV-2 but negative in the NAT test, is immediately eligible for plasma donation.

In some embodiments, if said donor is asymptomatic and is only tested positive for the NAT test, or is positive for the NAT test with a subsequent positive for antibodies anti-SARS-CoV-2, said donor is eligible for plasma donation 28 days after collecting the sample for the NAT test.

In some embodiments, if said donor is asymptomatic and is only positive for anti-SARS-CoV-2 antibodies, said donor is eligible for plasma donation after 7 days after the serology test.

In some embodiments, if said donor is asymptomatic and is positive for the NAT test with a subsequent negative for the NAT test, said donor is eligible for plasma donation 14 days after the negative NAT test.

In some embodiments, if said donor is symptomatic and is only positive for the NAT test, or is positive for the NAT test with a subsequent positive for anti-SARS-COV-2 antibodies, or is only positive for anti-SARS-COV-2 antibodies, or is positive for the NAT test with a subsequent negative for anti-SARS-COV-2 antibodies, said donor is eligible for plasma donation 28 days after cessation of all symptoms.

In some embodiments, if said donor is symptomatic and is positive for the NAT test with a subsequent positive for the NAT test, said donor is eligible for plasma donation 28 days after cessation of all symptoms or 28 days after the second NAT test, whichever is later.

In some embodiments, if said donor is symptomatic and is positive for the NAT test with a subsequent negative for the NAT test, said donor is eligible for plasma donation 14 days after cessation of all symptoms.

In some embodiments, said plasma is screened at least for blood-borne pathogens and blood type.

In some embodiments, said plasma is freezed after collection.

Hereinafter, the present invention is described in more detail with reference to illustrative examples, which does not constitute a limitation of the present invention.

### EXAMPLES

### Example 1

### Selection of Plasma Donors for Collection of SARS-CoV-2 Convalescent Plasma

For the selection of plasma donors for obtaining SARS-CoV-2 convalescent plasma for use in the production of the hyperimmune globulin composition of the present invention, the method described in U.S. 63/034,289 (incorporated by reference herein) is used.

In brief, individuals in good health who have been approved through the pre-screening process are allowed to proceed to the donation center for final evaluation and donation. This pre-screening process assured that only individuals who have recovered from their illness, or were exposed to the disease agent but remained asymptomatic, would qualify to come into the center and potentially donate. Thus, only individuals that had a laboratory evidence of COVID-19 infection, either through nucleic acid amplification testing (NAT), positive antigen test, or by SARS-CoV-2 antibody test prior to enrollment, and were then in a convalescent noninfectious state may be safely processed within the donor center.

Thus, symptomatic donors had to have complete resolution of symptoms at least 14 days before the donation if they were negative by a follow-up NAT, or 28 days if they had no follow-up test. Similarly, asymptomatic donors who were positive by NAT or antigen tests were required to wait 14 days after the initial test if they had a follow-up negative NAT, but had to wait 28 days after the initial test if they had no follow-up test. Asymptomatic donors who were only tested by an anti-SARS-CoV-2 antibody test were required to wait seven days prior to donation, but could donate immediately if they also had a negative NAT.

Donors also had to be negative for human leukocyte antigen (HLA) antibodies.

Table 1 summarizes the above criteria for plasma donors' eligibility based on symptoms and test results.

TABLE 1
Criteria for plasma donors' eligibility.

| | Test results | 1^{st} Eligible date |
|---|---|---|
| Symptomatic | No NAT, Negative | Not eligible, but could be |
| | Antibody | eligible for normal source |
| | | plasma |
| | Positive NAT only | 28 days after cessation of all |
| | Positive NAT with | symptoms. |
| | subsequent Positive | NOTE: The Positive Antibody |
| | Antibody | test indicates past infection |
| | Positive Antibody | with COVID 19; |
| | only | |
| | Positive NAT, | |
| | Antibody negative | |
| | Positive NAT, then | 28 days after cessation of all |
| | second Positive NAT | symptoms or 28 days after |
| | later | second positive at NAT test, |
| | | whichever is later |
| | Positive NAT with | 14 days after cessation of all |
| | subsequent Negative | symptoms. |
| | NAT | NOTE: The second negative |
| | | NAT test indicates that |
| | | the donor no longer has a |
| | | detectable amount of virus and |
| | | therefore the deferral period |
| | | may be shortened. |
| Asymptomatic | No NAT, negative | Eligible for Normal Source |
| | antibody | Plasma |
| | Negative NAT and | Eligible today |
| | Positive Antibody | |
| | if collected at | |
| | same time | |
| | Positive NAT only | 28 days after date of positive |
| | Positive NAT with | at NAT sample. Confirmation |
| | subsequent Positive | needed that subsequently, no |
| | Antibody | symptoms were developed. If |
| | | symptoms developed, reset the |
| | | calendar to a symptom-based. |
| | | NOTE: The Positive Antibody |
| | | test indicates past infection |
| | | with COVID 19 |
| | Positive Antibody | At least 7 days from the |
| | only | serology test result if no |
| | | symptoms were developed. |
| | Positive NAT with | 14 days after date of positive |
| | subsequent Negative | at NAT sample. |
| | NAT | NOTE: The second negative |
| | | NAT test indicates that the |
| | | donor no longer has a |
| | | detectable amount of virus and |
| | | therefore the deferral period |
| | | may be shortened. |
| | Positive antigen | 28 days after date of positive at |
| | only | NAT sample. Confirmation |
| | | needed that subsequently, no |
| | | symptoms were developed. |
| | Positive antigen | 14 days after date of positive |
| | with subsequent | at NAT sample. |
| | Negative NAT | |

### Example 2

### Manufacture of SARS-CoV-2 Convalescent Human Plasma

Once the donor has been selected as explained in example 1 or following any other criteria, plasma is collected by plasmapheresis.

Each plasma unit must meet requirements for source plasma for manufacturing as defined by regulations including screening against a variety of infectious agents. Additionally, each unit was tested to confirm it was negative for SARS-CoV-2 virus and positive for anti-SARS-CoV-2 antibodies.

Each plasma sample was also tested to be negative for human leukocyte antigen (HLA) antibodies and blood typed. Then, plasma pools were modeled to maintain consistent distribution with the overall donor ABO blood type distribution to maintain consistent batch to batch levels of anti-A and anti-B.

These parameters are normally limited by dilution when large batches of plasma are pooled together to make immunoglobulin products, but with smaller batches, single donors could have a greater influence on the final product.

Thus, type 0 and Type B donors were limited to no more than two units from any single donor for each plasma pool to decrease the likelihood of having high anti-A titers in the final product.

In this example, the ABO blood typing results from 500 plasma units used for manufacturing the pool batches of SARS-CoV-2 convalescent human plasma are presented in Table 2. Results from two published studies are included as comparators. These results show that ABO blood type distribution for the COVID-19 convalescent plasma donors was similar to the distributions reported in other studies of blood and plasma donors.

TABLE 2
ABO Blood type distribution of convalescent plasma from test
batches for this invention compared to published values.

ABO Type (%)

| | A | B | O | AB |
|---|---|---|---|---|
| This invention (500 Units) | 36.4 | 9.0 | 48.6 | 6.0 |
| Garratty et al, (2004) | 37.1 | 12.2 | 46.6 | 4.1 |
| McVey et al (2015) | 38.0 | 11.3 | 47.1 | 3.7 |

### Example 3

### Manufacture of a Liquid Therapeutic Hyperimmune Globulin Composition from SARS-CoV-2 Convalescent Plasma

The plasma pools obtained in the example 2 were then processed following the same steps as the Gamunex-C caprylate/chromatography process (Lebing, W., et al., 2003, U.S. Pat. No. 6,307,028, each incorporated by reference herein), which included multiple steps validated for the removal and/or inactivation of viruses (Gamunex-C [Immune Globulin Injection (Human) 10% Caprylate/Chromatography Purified]-Package Insert. 2020).

The resulting product was a highly purified IgG solution (SARS-CoV-2 human immunoglobulin (hIVIG)) formulated at around 10% protein content with glycine at a low pH.

### Example 4

### Characterization of SARS-CoV-2 Human Immunoglobulin (hIVIG) Product

The hyperimmune globulin composition of the present invention (hIVIG), obtained from SARS-CoV-2 convalescent human plasma, was characterized to assess the recovery of anti-SARS-CoV-2 specific antibodies. Thus, hIVIG product was tested with an IgG specific Enzyme-linked immunosorbent assay (ELISA) and a neutralizing antibody assay.

Characterization of hIVIG product also included prior routine batch testing to characterize the product and ascertain that it is suitable for use. This characterization included analyses for glycine, pH, protein concentration, osmolality, composition by electrophoresis, and molecular weight profiling by size exclusion chromatography. Analyses were also performed for sodium caprylate, residual IgA and IgM, prekallikrein activator (PKA), factor Xa, anti-A, anti-B, and anti-D. In addition, compendial tests for sterility and pyrogenic substances were performed on all batches.

These tests showed that the tested batches were within the batch standards for purity, formulation, molecular profile and purity described for other immune globulin products manufactured with the caprylate/chromatography process, such as Gamunex-C. The batches also passed USP pyrogen and sterility tests.

Surprisingly, these tests showed that between 97% and 100% of the protein content was IgG. In addition, the IgG was present almost entirely as monomers and dimers with aggregates and fragments below the limits of detection. A process impurity (sodium caprylate) and plasma protein impurities were found at very low concentrations in the final product, well under the batch requirements.

The amounts of residual IgA and IgM were also below the batch requirements (less than 0.13 mg/ml and less than 0.030 mg/ml, respectively) and the concentrations known for the Gamunex-C product.

IgM has been identified as a primary source of anti-A and anti-B intravascular hemolytic activity (Flegel, W. A., 2015). The hIVIG product of the present invention was shown to contain less than 0.01 mg/ml, which greatly reduces the danger of this adverse event. In contrast, when patients are treated with convalescent plasma, they must be matched by donor blood type to reduce the chances of hemolysis.

Similarly, removal of IgA provides a potential therapeutic advantage for hIVIG products over convalescent plasma in patients who are IgA deficient and may have been previously treated with blood products and formed antibodies to IgA. The hIVIG product of the present invention was shown to contain less than 0.04 mg/ml of IgA.

### Anti-SARS-CoV-2 ELISA

Anti-SARS-CoV-2 IgG titers were determined using Human Anti-SARS-CoV-2 Virus Spike 1 (S1) IgG assay from Alpha Diagnostic. 20 hIVIG batches were tested using multiple serial dilutions and a curve constructed by plotting the log of the optical density as a function of the log of the dilution. The titer was defined as the dilution at which this curve is equal to the low kit standard. The titer was also expressed as a ratio to an in-house control, which consists of a commercially available chimeric monoclonal SARS-CoV-2 S1 antibody (Sino Biologicals, Beijing, China) spiked into plasma from non-COVID-19 donors at levels intended to give titers similar to those found in plasma of COVID-19 donors.

The results are presented in Table 3 for the 20 hIVIG batches produced and its corresponding plasma pool. Said results demonstrated that ELISA activity (ELISA titer, 1:X) increased up to almost 30-fold, when processing the pooled plasma into the final product. The IgG concentration was also increased more than 10-fold from the pooled plasma to the final product. When anti-SARS-CoV-2 antibody titers were normalized to the IgG concentration, data varies between 250 and 2,500, which result in similar values for the starting material and the finished product. This can be explained by contributions from IgM and IgA to the ELISA activity, which have been removed during purification of IgG and demonstrates once again the high purity of the hIVIG final product.

TABLE 3
Anti-SARS-CoV-2 titers and specific activities
(n = 20, ±standard deviation).

| | | | | | Normalized |
|---|---|---|---|---|---|
| | | | Normalized | Neutralizing | Neutralizing |
| | | | ELISA | Activity | Activity, |
| Sample | IgG, | ELISA | Titer, titer/ | Titer, | IC50/mg/mL |
| Point | mg/mL | Titer, 1:X | mg/mL IgG | IC50, 1:X | IgG |
| Pool | 8.0 ± | 9,038 ± | 1,131 ± | 110 ± | 13.9 ± |
| | 0.2 | 4037 | 497 | 15 | 2.2 |
| Final | 100 ± | 25,000- | 250- | 150- | 1.5- |
| Product | 3 | 250,000 | 2,500 | 1,500 | 15 |
| (n = 20) | | | | | |

### Anti-SARS-CoV-2 Neutralizing Antibody Assay

The hIVIG products were also tested for anti-SARS-CoV-2 antibodies using an immunofluorescence-based neutralization assay performed at the National Institutes of Health Integrated Research Facility, Frederick, Md. This assay quantifies the anti-SARS-CoV-2 neutralization titer by using a dilution series of test material to test for inhibition of infection of cultured Vero (CCL-81) cells by SARS-CoV-2 (Washington isolate, CDC).

Potency was assessed using a cell-based immunosorbent assay to quantify infection by detecting the SARS-CoV-2 nucleoprotein using a specific antibody raised against the SARS-CoV-1 nucleoprotein.

The secondary detection antibody was conjugated to a fluorophore which allows quantification of individual infected cells on a high throughput optical imaging system. A minimum of 16,000 cells were counted per sample dilution across four wells—two each in duplicate plates. Data are reported based on a 4-parameter regression curve (using a constrained fit) as a 50% neutralization titer (IC50) in Table 3.

The results showed that antibody neutralizing activity (IC50) was increased more than 10-fold from the plasma pool to the final product. This increase in neutralizing activity indicates that patients treated with hIVIG products compared to an equivalent volume of convalescent plasma would receive higher neutralizing activity. Alternatively, patients treated with hIVIG could receive a smaller treatment volume compared to treatment with convalescent plasma and potentially decrease the chances for transfusion-associated circulatory overload.

Specific neutralizing activity (normalized to the IgG concentration), was slightly reduced in final product compared to plasma. As previously discussed, this may be caused by contributions from IgM and IgA which have been removed during purification of IgG.

An advantage of using SARS-CoV-2 convalescent human plasma to manufacture the hIVIG product of the present invention (compared to direct administration of plasma from individuals or administration of a monoclonal antibody) is the diversity of antibodies obtained from a pool of convalescent donors which may provide a wider range of anti-viral activity. This diversity is important in overcoming mutations in the virus. Antibody diversity provides a broader range of anti-viral activity by attacking different viral epitopes and enlisting different cellular mechanisms. Neutralization of free virus is mainly the result of steric blocking to prevent infection, whereas additional anti-viral activity may come from activation of effector functions such as complement-mediated or antibody-dependent cellular cytotoxicity.

### Example 5

### Manufacture of a Liquid Therapeutic Hyperimmune IgM Composition from SARS-CoV-2 Convalescent Plasma

The plasma pools obtained in the example 2 were processed in a similar way as the

Gamunex process. The eluate of Q-Sepharose anion exchange (Q-strip) had the higher IgM titres compared to that of the ANX strip, and was then processed following the steps of:
a) precipitation of said IgM using PEG-3350 at 10% (w/w) at a pH of 5.0-6.0;
b) resuspension of the precipitated IgM in a buffer comprising 5 mM sodium phosphate, 20 mM tris, 1 M NaCl pH 8.0;
c) adsorption chromatography using a ceramic hydroxyapatite (CHT) chromatography;
d) isoagglutinin affinity chromatography;
e) nanofiltration; and
f) ultrafiltration/diafiltration.

The obtained product showed typical immunoglobulin levels for IgG, IgA, and IgM as shown in Table 4, as well as their content in the obtained product.

TABLE 4
Content of IgA, IgG and IgM in the final IgM
bulk. IgM Clin Avg refers to the average of
4 larger scale runs from pre-pandemic plasma.

| | IgA | IgG | IgM | IgA | IgG | IgM |
|---|---|---|---|---|---|---|
| IgM Batch | (mg/ml) | (mg/ml) | (mg/ml) | (%) | (%) | (%) |
| Batch 2 | 1.19 | 0.44 | 30.2 | 3.7 | 1.4 | 94.9 |
| Batch 3 | 2.12 | 2.16 | 27.1 | 3.8 | 6.9 | 86.4 |
| Batch 4 | 1.15 | 0.58 | 30.1 | 3.6 | 1.8 | 94.6 |
| IgM Clin Avg | 0.49 | 0.10 | 32.2 | 1.6 | 0.3 | 98.1 |

The pooled plasma (Q-strip) and IgM bulks were tested for binding to the S1 protein of the SARS-CoV-2 antigen, and the results showed an approximate 35-40 increase in IgM antigen binding to SARS-CoV-2 S1 protein in the bulk as compared to that of the pooled plasma. This is shown in Table 5.

TABLE 5
Titre anti S1 protein of SARS-CoV-2 antigen in the starting pooled plasma and in the final
IgM bulk. IgM Pre-pandemic refers to a bench scale IgM prepared from pre-pandemic plasma.

| | Convalescent Plasma Pool | | Q Strip | | IgM bulk | |
|---|---|---|---|---|---|---|
| IgM Batch | Titre | Ratio to plasma | Titre | Ratio to plasma | Titre | Ratio to plasma |
| Batch 2 | 203.0 | 1.0 | 282.6 | 1.4 | 7210.4 | 35.5 |
| Batch 3 | 357.5 | 1.0 | 512.1 | 1.4 | 12357.4 | 34.6 |
| Batch 4 | 103.1 | 1.0 | 181.8 | 1.8 | 4208.0 | 40.8 |
| IgM Pre-pandemic | <100 | N/A | <100 | N/A | 385 | N/A |

### Example 6

### Characterization of SARS-CoV-2 Human Immunoglobulin M Product

Characterization of the IgM product included prior routine batch testing to characterize the product and ascertain that it is suitable for use.

Table 6 below shows the IgM profile. Aggregate, oligomer, pentamer and <pentamer were identified by SEC-HPLC.

TABLE 6
IgM profile in the final bulk. IgM Clin Avg refers to the
average of 4 larger scale runs from pre-pandemic plasma.

| IgM | Aggregate | Oligomer | Pentamer | <Pentamer |
|---|---|---|---|---|
| Batch | (%) | (%) | (%) | (%) |
| Batch 2 | 0.4 | 4.1 | 94.6 | 0.9 |
| Batch 3 | 11.1 | 12.4 | 75.6 | 0.9 |
| Batch 4 | 0.5 | 4.5 | 94.1 | 0.9 |
| IgM Clin | 0.5 | 2.4 | 96.7 | 0.5 |
| Avg | | | | |

The IgM bulk also showed a reactive signal to all of the Maverick™ (Genalyte Inc., USA) SARS-CoV-2 protein panel (18-97 fold compared to each batch of convalescent pooled plasma), as shown in Tables 7 and 8. The Maverick SARS-CoV-2 Multi-Antigen Serology Panel v2 is a photonic ring immunoassay (PRI) intended for qualitative detection of total antibodies (including IgG and IgM) to SARS-CoV-2 in human dipotassium EDTA plasma using the Maverick™ Diagnostic System.

TABLE 7
Results of IgM bulk to antigens from SARS-CoV-2.

| | | CoV-2 | CoV-2 | CoV-2 | CoV-2 |
|---|---|---|---|---|---|
| | CoV-2 | Spike | Spike | Spike | Spike |
| IgM Batch | nucleocapsid | S1 | S1 RBD | S1S2 | S2 |
| Batch 2 | 293.5 | 561.35 | 2218.75 | 353.03 | 452.9 |
| Batch 3 | 237.9 | 462.98 | 1696.15 | 211.10 | 229.7 |
| Batch 4 | 166.3 | 281.15 | 1124.65 | 178.13 | 218.58 |
| Non-reactive | 21 | 9.7 | 49 | 75 | 37 |
| cut-off | | | | | |

TABLE 8
IgM results ratio to each batch of convalescent plasma pool.

| | | CoV-2 | CoV-2 | CoV-2 | CoV-2 |
|---|---|---|---|---|---|
| | CoV-2 | Spike | Spike | Spike | Spike |
| IgM Batch | nucleocapsid | S1 | S1 RBD | S1S2 | S2 |
| Batch 2 | 46.6 | 27.4 | 31.2 | 26.7 | 31.9 |
| Batch 3 | 97.9 | 25.5 | 26.5 | 28.0 | 35.0 |
| Batch 4 | 22.8 | 18.0 | 35.7 | 20.3 | 19.4 |

### Anti-SARS-CoV-2 Neutralizing Antibody Assay

The IgM products were tested for anti-SARS-CoV-2 antibodies using different laboratories and different technique, as shown in Table 9, to three different batches. Laboratory 1 used the Cytopathic-Cytotoxicity Luminometry Assay (COLA); Laboratory 2 used the Plaque Forming Units (PFU) technique, while Laboratory 3 used the Median Tissue Culture Infectious Dose (TCID₅₀).

In all cases, the half-maximal inhibitory concentration (IC50) was calculated as immunoglobulin dilution.

TABLE 9
SARS-CoV-2 infectivity neutralization by hyperimmune
IgM composition of the present invention.

Laboratory:

| | Lab 1 | Lab 2 | Lab 3 | Lab 3 |
|---|---|---|---|---|
| | May 10, | Sep. 11, | Sep. 12, | Apr. 2, |
| | 2020 | 2020 | 2020 | 2021 |
| Infectivity | CCL | PFU | TCID₅₀ | TCID₅₀ |
| Neutralization | | | | |
| assay | | | | |
| Batch 2 | 8182 | 14791 | NT | NT |
| Batch 3 | 62425 | 58884 | 2263 | 320 |
| Batch 4 | NT | NT | 1132 | 247 |

An advantage of using convalescent human plasma to manufacture the IgM product of the present invention (compared to direct administration of plasma from individuals or administration of a monoclonal antibody) is the diversity of antibodies obtained from a pool of convalescent donors which may provide a wider range of anti-viral activity. This diversity is important in overcoming mutations in the virus. Antibody diversity provides a broader range of anti-viral activity by attacking different viral epitopes and enlisting different cellular mechanisms. Neutralization of free virus is mainly the result of steric blocking to prevent infection, whereas additional anti-viral activity may come from activation of effector functions such as complement-mediated or antibody-dependent cellular cytotoxicity.

1. A liquid therapeutic hyperimmune globulin composition comprising human plasma-derived immunoglobulin G (IgG) with a purity of at least 97% of the total protein content and having a SARS-CoV-2 antibody titre between 250 and 2,500 per mg/mL of IgG and/or a SARS-CoV-2 neutralization activity (IC50 neutralization titer) between 1.5 and 15 per mg/mL of IgG.

2. The composition according to claim 1, wherein the purity of said human plasma-derived IgG is at least 98%, of the total protein content.

3. The composition according to claim 1, wherein the SARS-CoV-2 antibody titre is between 300 and 2,200 per mg/mL of IgG.

4.-8. (canceled)

9. The composition according to claim 1, wherein the SARS-CoV-2 neutralization activity is between 1.8 and 12 per mg/mL of IgG.

10.-13. (canceled)

14. The composition according to claim 1, wherein the human plasma-derived IgG content is between 5% and 20% (w/v).

15. (canceled)

16. The composition according to claim 1, wherein at least 90% of the human plasma-derived IgG is present as monomers and dimers.

17. The composition according to claim 1, wherein the content of immunoglobulin A (IgA) is equal or lower than 0.04 mg/ml.

18. The composition according to claim 1, wherein the content of immunoglobulin M (IgM) is equal or lower than 0.01 mg/ml.

19. A liquid therapeutic hyperimmune globulin composition comprising human plasma-derived immunoglobulin G (IgG) with a purity of at least 97% of the total protein content and having a SARS-CoV-2 antibody titre between 25,000 and 250,000 and/or a SARS-CoV-2 neutralization activity (IC50 neutralization titer) between 150 and 1,500.

20. The composition according to claim 19, wherein the purity of said human plasma-derived IgG is at least 98% of the total protein content.

21. The composition according to claim 19, wherein the SARS-CoV-2 antibody titre is between 50,000 and 200,000.

22.-24. (canceled)

25. The composition according to claim 19, wherein the SARS-CoV-2 neutralization activity is between 200 and 1,250.

26.-29. (canceled)

30. The composition according to claim 19, wherein the human plasma-derived IgG content is between 5% and 20% (w/v).

31. (canceled)

32. The composition according to claim 19, wherein at least 90% of the human plasma-derived IgG is present as monomers and dimers.

33. The composition according to claim 19, wherein the content of immunoglobulin A (IgA) is equal or lower than 0.04 mg/ml.

34. The composition according to claim 19, wherein the content of immunoglobulin M (IgM) is equal or lower than 0.01 mg/ml.

35. (canceled)

36. A method for preparing the liquid therapeutic hyperimmune globulin composition according to claim 1, from a starting solution comprising anti-SARS-CoV-2 IgG antibodies, the method comprising the sequential a) through e) of:
a) adjusting the pH of the starting solution to be within a range of from about 3.8 to about 4.5 to form an intermediate solution comprising dissolved antibodies,
b) adding a source of caprylate ions to the intermediate solution of said a) and adjusting the pH of the intermediate solution to be within a range of from about 5.0 to about 5.2 to form a precipitate and a supernatant solution comprising dissolved antibodies,
c) incubating the supernatant solution under conditions of time, temperature and caprylate ion concentration to inactivate substantially all viruses,
d) contacting the supernatant solution with at least one ion exchange resin under conditions that allow binding of at least some of the other substances including IgA or IgM to the resin while not allowing binding of the antibodies including IgG to the resin, and
e) collecting the IgG antibodies,
wherein the starting solution is SARS-CoV-2 convalescent human plasma.

37. The method according to claim 36, wherein the SARS-CoV-2 convalescent human plasma is a pool of plasma samples from at least two convalescent donors.

38. The method according to claim 36, wherein the SARS-CoV-2 convalescent human plasma is tested negative for at least one of blood-borne pathogens and human leukocyte antigen (HLA) antibody.

39. The method according to claim 36, wherein the SARS-CoV-2 convalescent human plasma is tested for blood type.

40.-67. (canceled)
